(19) United States
(12) Patent Application Publication
(10) Pub. No.: US 2003/0060845 A1
(43) Pub. Date: Mar. 27, 2003

(54) Tubular compression orthosis for immobilising a lower limb after a venous surgical procedure

(76) Inventor: Christian Gardon-Mollard, Chamalieres (FR)

Correspondence Address: JACOBSON HOLMAN PLLC, 400 SEVENTH STREET N.W., SUITE 600, WASHINGTON, DC, 20004, US

(21) Appl. No.: 10/111,553

(22) PCT Filed: Aug. 3, 2001
(86) PCT No.: PCT/FR01/02541

(30) Foreign Application Priority Data
Sep. 4, 2000 (FR) 00/11240

(51) Int. Cl. A61F 13/00; A61F 13/06; A61B 17/00

(52) U.S. Cl. 606/201; 602/62; 602/63

(57) ABSTRACT

The orthosis comprises a compressive tubular portion (10) of knitted or woven fabric, of varying section that matches the profile of the lower limb, and in which the knit or weave structure is selected as a function of the peripheral dimensions of the lower limb so as to apply compression pressure of about 30 mmHg to 40 mmHg that is substantially constant along the length of the limb. The tubular portion extends from the foot to the top of the thigh and comprises a stretchable inner region (12) that is highly elastic and that is connected along two generator lines to an outer region (14) that is non-stretchable or poorly stretchable. The top end of the orthosis is also provided with a thigh sleeve (22) that is open along a generator line and that is provided with adjustable closure means (24).

The invention relates to the field of compressive orthoses for use in the immediate postoperative period after surgery of veins in the lower limbs.

Surgery of superficial veins comprises two main operations, both of which involve surgical removal of the diseased vein.

The first operation is known as “stripping” which comprises tearing out the vein axis of the inner or outer saphena on a wire. This operation is performed under an anesthetic which can be general, loco-regional, peridural, or under neuroleptanalgesia. It gives rise to significant damage to the collateral branches of the saphena which consequently bleed, giving rise to hematomas, which are painful and uncomfortable for patients in the hours and days following surgery.

The other technique in common use is outpatient phlebectomy, which is a surgical act that is less traumatizing than stripping (but which can also be performed in the context of stripping). This technique involves thorough exeresis of the superficial veins under outpatient conditions. The hematomas are smaller than with stripping, but the risk of immediate bleeding exists when the patient gets up, such risks being inherent to outpatient surgery.

In both cases, in order to reduce bleeding and attenuate the consequences thereof, immediately after surgery the surgeon applies compression very strongly to the operated limb so as to limit postoperative inflammation and bleeding.

In nearly all cases, such compression is applied at present using dry or adhesive bands.

That technique presents several drawbacks:

by its very nature it depends on the skill of the person performing it;

it is not possible to monitor the value and the uniformity of the pressure applied: as a result some bands run the risk of being too tight and therefore painful, or else not tight enough and therefore ineffective.

In practice, during the period immediately following surgery, it is found that patients generally suffer far more from bands that have been poorly put into place than they do from the operation itself.

Other methods of vascular surgery give rise to discomfort of the same kind, for example methods referred to as “conservative surgery” derived from conservative hemodynamic outpatient treatment for vein insufficiency (known in French under the initials CHIVA).

One of the objects of the invention is to propose an orthosis which, on being put into place immediately after an operation, serves to provide controlled and regular compression over the entire lower limb in a manner that does not depend on the skill of the person applying the orthosis, and that compresses sufficiently to limit postoperative inflammation and bleeding.

The bands that are currently in widespread use constitute the orthosis that is easiest to put into place, but as mentioned above, that is least suited to the purpose of applying compression.

It is possible, instead, to use some other existing type of orthosis, in particular medical elastic stockings. However such stockings apply pressure degressively, and in particular they apply little pressure to the thigh, whereas under postoperative conditions, it is specifically on the thigh where the pressure needs to be applied most strongly. Furthermore, such stockings are difficult to put on a patient who is unconscious because of general anesthesia and also because of the dressings that have been applied to the leg and that increase its volume.

The orthosis of the invention serves to mitigate all of those drawbacks.

Thus, the invention provides a tubular compressive orthosis for compressing a lower limb after vein surgery, in particular after outpatient phlebectomy or vein stripping, which orthosis comprises a compressive tubular portion of knitted or woven fabric, and of varying section matching the profile of the lower limb, and in which the structure of the knit or the weave is selected as a function of the peripheral dimensions of the lower limb so as to apply compression at a pressure which is substantially constant along the length of the limb.

According to various advantageous secondary characteristics:

the substantially constant compression pressure lies in the range 30 millimeters of mercury (mmHg) to 40 mmHg;

the orthosis is in the form of an open stocking, having no knitted heel or foot;

the tubular portion extends from the foot to the top of the thigh;

the tubular portion comprises a stretchable inner region that is highly elastic and that is connected along two generator lines, e.g. approximately diametrically opposite generator lines, to an outer region that is non-stretchable or poorly stretchable;

the top end of the tubular portion comprises a thigh sleeve that is open along a generator line and that is provided with adjustable closure means; and

the top and/or bottom ends of the orthosis are made of material that can be cut and that is run-resistant, so as to enable the orthosis to be cut to length when it is put into place.

The invention is described below in greater detail with reference to the accompanying drawings.

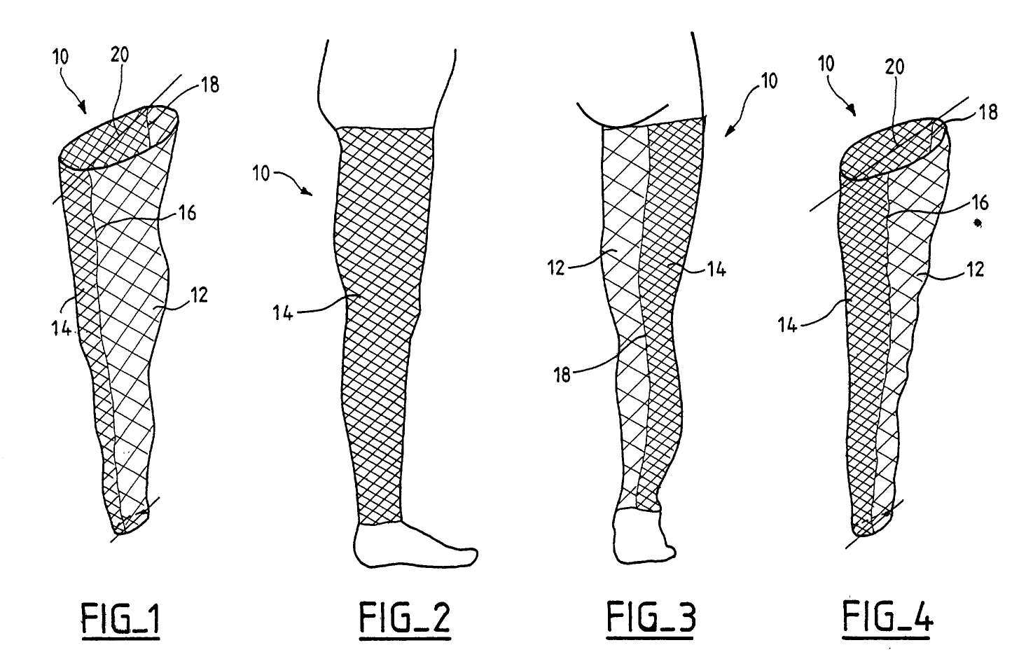

FIG. 1 shows an orthosis of the invention prior to being put into place.

FIGS. 2 and 3 are respectively a side view and a back view of the FIG. 1 orthosis in place on a lower limb.

FIG. 4 shows a variant of the FIG. 1 orthosis.

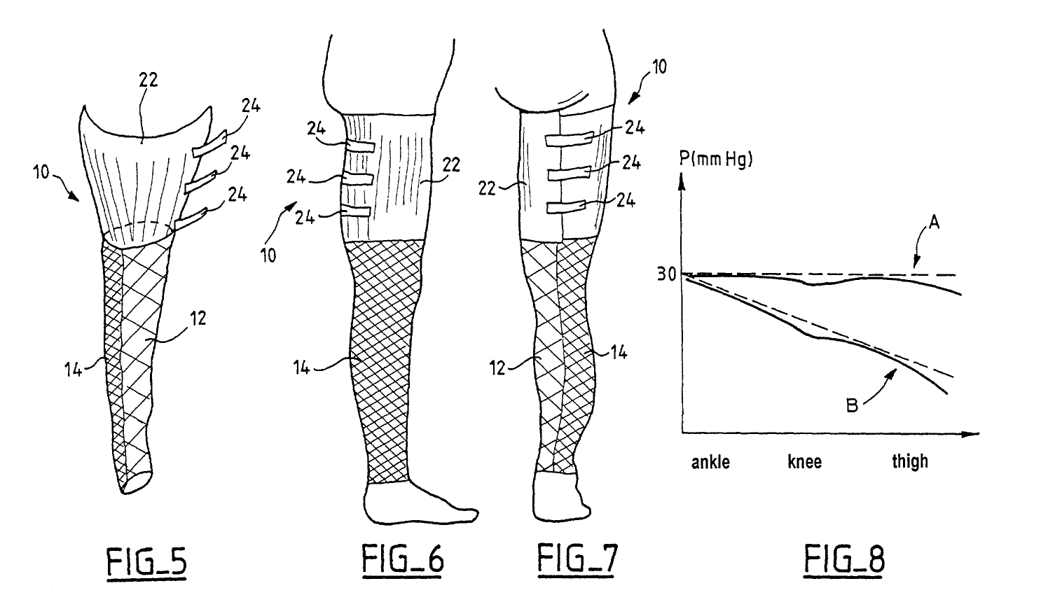

FIG. 5 shows an improvement applied to the FIG. 1 orthosis.

FIGS. 6 and 7 are respectively a side view and a back view of the improved orthosis of FIG. 5 put into place on a lower limb.

FIG. 8 is a graph showing the respective compression pressure profiles as obtained by an orthosis of the invention and by a conventional elastic stocking.

FIG. 1 shows the orthosis of the invention which is essentially in the form of a stocking that is open at the foot. More precisely, the orthosis 10 is in the form of a knitted tube of varying section that matches the shape of a leg, i.e. it tapers downwards, and it advantageously does not have a knitted foot or heel.

This orthosis is for covering the lower limb, as shown in FIGS. 2 and 3, from the foot to the top of the thigh.

It must be capable of exerting constant compressive pressure on the lower limb from the ankle to the thigh (unlike traditional elastic stockings which produce pressure that decreases going away from the ankle), and this pressure should be about 30 mmHg to 40 mmHg (40.0 hectopascals (hPa) to 53.2 hPa), thus enabling a regular and constant hemostatic effect to be exerted all along the lower limb.

The tubular orthosis 10 comprises two substantially semicylindrical portions 12 and 14 which extend along the entire length of the orthosis and which are connected together along two generator lines 16, 18 situated at opposite ends of a diameter 20.

The semicylindrical portion 12 that is situated on the inside of the leg is constituted by a reinforced elastic stitch, e.g. of the same type as that used on the seat face of pantyhose.

The semicylindrical portion 14 situated on the outside of the leg is knitted using a stitch that stretches little, i.e. a stitch with short elongation, so as to apply greater compression on the outer face.

The term “elasticity” is used herein in the sense that is conventional for textiles, i.e. to denote the ability of a textile material that has been stretched to return to its initial shape and dimensions once the deforming forces have been removed; this elasticity is expressed in percentage elongation relative to one meter at rest.

Elongation is said to be “short” (for a stitch that stretches little, such as that used in the portion 14) when maximum elongation is less than 70% (see in particular C. Gardon-Mollard and A.-A. Ramelet in “La Contention Médicale” [Medical compression], published by Masson, Paris, 1999, pp. 51-54) and elongation is said to be “medium” (an “elastic” stitch such as that used in the portion 12) when the maximum elongation lies in the range 70% to 140%.

Advantageously, the orthosis is manufactured in several sizes, typically three or four sizes of different diameters but having a single length, with the orthosis being fitted to the length of the patient's leg by cutting off excess length at each end. The orthosis should then be knitted, at least at both ends, with a stitch of the run-resistant type so as to avoid unraveling after it has been cut to length.

The stitch can be of the same type as is used in a conventional elastic stocking, e.g. the Varisma (registered trademark) stocking produced by Innothéra Topic. It can be of the wefted, plain, pinched or floated micromesh, etc. type, all of which stitches are known per se to specialists in knitting techniques.

The yarn used for knitting the orthosis can be an elastane covered in cotton and polyamide, an elastane covered in polyamide without any cotton, or indeed a mixture of elastane and elastodiene (synthetic rubber latex). Nevertheless, the material advantageously includes a large fraction of cotton so as to be more effective in absorbing serosites and bleeding; it can also be selected so as to be washable and optionally reusable.

The orthosis of the invention is advantageously put into place using an accessory of the kind described in WO-A-99/44558 (Innothéra Topic International) which makes putting on and adjusting the orthosis simple, quick, and accurate, even in the presence of thick dressings and on a limb that is still anesthetized.

Naturally, a variety of variants can be envisaged.

In a first variant shown in FIG. 4, the outer semicylindrical portion 14 is of greater peripheral extent than the elastic inner portion 12, i.e. the two generator lines 16 and 18 where the portions are joined together both lie on the same side of a diameter 20.

Another variant consists not in knitting the orthosis but in making it up from two kinds of woven fabric presenting the same deformation characteristics as those described above, i.e. both a fabric having small elongation for the outer face 14 and an elastic fabric for the inner face 12. To avoid the drawbacks associated with lines of stitching, the two semicylindrical portions can be connected together using textile heat-sealing or high frequency methods, which are known per se.

An improvement shown in FIGS. 5 to 7 consists in providing the top portion of the orthosis in the form of a thigh sleeve that is not elastic but that is adjustable, e.g. made of woven fabric.

To make it easy to put on, this sleeve is open along a generator line as shown in FIG. 5. After the orthosis has been put on the leg, the practitioner presses the sleeve 22 against the top portion of the thigh and moves its two free edges towards each other by adjusting the tightening so as to apply the desired amount of compression to this region of the lower limb. The thigh sleeve 22 is then closed in the desired position, e.g. by means of self-fastening closure strips 24 which make it easy to adjust tightness, where necessary.

FIG. 8 is a graph showing the compression profile that is obtained using the orthosis of the invention, i.e. substantially constant profile A, in comparison with the degressive profile B that is obtained using a conventional elastic stocking. The dashed-line profiles are ideal profiles, while the continuous-line profiles represent real measurements. These measurements take account both of the shape of the knee which locally prevents regular pressure being applied, and also the fact that it is difficult to maintain pressure at the top of the thigh because of the large diameter of this region (Laplace's law).

1. A tubular compressive orthosis for compressing a lower limb after vein surgery, in particular after outpatient phlebectomy or vein stripping, the orthosis being characterized in that it comprises a compressive tubular portion (10) of knitted or woven fabric, and of varying section matching the profile of the lower limb, and in which the structure of the knit or the weave is selected as a function of the peripheral dimensions of the lower limb so as to apply compression at a pressure which is substantially constant along the length of the limb.

2. The orthosis of claim 1, in which the tubular portion (10) comprises a stretchable inner region (12) that is highly elastic and that is connected along two generator lines (16, 18) to an outer region (14) that is non-stretchable or poorly stretchable.

3. The orthosis of claim 2, in which the two generator lines (16, 18) interconnecting the inner and outer regions are approximately diametrically opposite.

4. The orthosis of claim 1, in which the substantially constant compression pressure lies in the range 30 mmHg to 40 mmHg.

5. The orthosis of claim 1, characterized in that it is in the form of an open stocking, having no knitted heel or foot.

6. The orthosis of claim 1, in which the tubular portion extends from the foot to the top of the thigh.

7. The orthosis of claim 1, in which the top end of the tubular portion comprises a thigh sleeve (22) that is open along a generator line and that is provided with adjustable closure means (24).

8. The orthosis of claim 1, in which the top and/or bottom ends of the orthosis are made of material that can be cut and that is run-resistant, so as to enable the orthosis to be cut to length when it is put into place.
